(19) United States
(12) Patent Application Publication
(10) Pub. No.: US 2024/0108613 A1
(43) Pub. Date: Apr. 4, 2024

(54) TOPICAL COMPOSITION FOR TREATING FUNGI FORMING ON A USER'S BODY

(71) Applicant: ACP LABORATORIES LLC, Hallandale, FL (US)

(72) Inventor: Lacramioara Pirvan, Hallandale, FL (US)

(73) Assignee: ACP LABORATORIES LLC, Hallandale, FL (US)

(21) Appl. No.: 18/276,222

(22) PCT Filed: Feb. 4, 2022
(86) PCT No.: PCT/US22/15381

§ 371 Date: Aug. 7, 2023

Related U.S. Application Data
(60) Provisional application No. 63/146,218, filed on Feb. 5, 2021.

(51) Int. Cl. A61K 31/454; A61K 31/14; A61K 33/40; A61K 36/45; A61K 47/44

(52) U.S. Cl. CPC A61K 31/454; A61K 31/14; A61K 33/40; A61K 36/45; A61K 47/44

(57) ABSTRACT

A topical composition for treating fungi forming on a user's body that includes a composition base forming 20-50% of the topical composition and selected from either or both benzethonium chloride at 0.2% concentration and 2-Octyl cyanoacrylate, and also includes isopropyl alcohol at 70% concentration and hydrogen peroxide at 3% concentration, or a combination of the above. The remaining components of the composition mixture may include apple cider vinegar, tea tree oil, fragranced bleach, and/or other components and ingredients that are non-active fillers, binders, or agents.

### FIELD OF THE INVENTION

The present invention relates generally to a composition and method of treating fungi and, more particularly, relates to a composition and method of treating fungi forming on a user's foot and/or nails.

### BACKGROUND OF THE INVENTION

Many users experience pain, discomfort and embarrassment caused by fungi forming on their bodies, in particular around the nails and nail beds of the body. Fungal infections, also known as mycosis, is disease caused by fungi. Different types of infections are traditionally divided according to the part of the body affected, i.e., superficial, subcutaneous, and systemic. Superficial fungal infections include common tinea of the skin, such as tinea of the body, groin, hands, feet and beard, and yeast infections such as pityriasis versicolor. Subcutaneous types include eumycetoma and chromoblastomycosis, which generally affect tissues in and beneath the skin. Systemic fungal infections are more serious and include cryptococcosis, histoplasmosis, pneumocystis pneumonia, aspergillosis and mucormycosis. Signs and symptoms range widely. There is usually a rash with superficial infection. Fungal infection within the skin or under the skin may present with a lump and skin changes. Pneumonia-like symptoms or meningitis may occur with a deeper or systemic infection. Fungi are everywhere, but only some cause disease. Fungal infection occurs after spores are either breathed in, come into contact with skin or enter the body through the skin such as via a cut, wound or injection.

The present invention is principally directed toward treating fungi forming and causing superficial infections and therefore principally has topical or transdermal application. Some very common treatments for fungi include the application of antifungal creams such as clotrimazole or miconazole. The known treatments, however, are problematic for many users having sensitive skin and/or that otherwise desire a less harsh reaction upon application of the treatments. Furthermore, experimental testing has shown that the present invention is more effective in preventing the recurrence of fungi, a problem experienced and not addressed by many of the known treatments.

Therefore, a need exists to overcome the problems with the prior art as discussed above.

### BRIEF DESCRIPTION OF THE DRAWINGS

There are no accompanying figures, as it is believed an understanding of invention is not currently served or furthered by the same. However, applicant reserves the right to add figures to illustrate potential embodiments of the present invention in subsequent applications.

### SUMMARY OF THE INVENTION

The invention provides a topical composition for treating fungi forming on a user's body that overcomes the hereinafore-mentioned disadvantages of the heretofore-known devices and methods of this general type and that provides safe, effective, and pain-less or pain-free treatment of areas on a user's body affected or having fungi.

With the foregoing and other objects in view, there is provided, in accordance with the invention, a topical composition for treating fungi forming on a user's body that includes a composition base forming 20-50% of the topical composition and selected from either or both benzethonium chloride at 0.2% concentration and 2-Octyl cyanoacrylate.

In accordance with another feature, an embodiment of the present invention includes approximately 50% efinaconazole at 5-10% concentration.

In accordance with yet another feature, an embodiment of the present invention also includes the topical composition having 60% efinaconazole at 5-10% concentration and with the composition base forming 40% of the topical composition.

In accordance with an additional feature, an embodiment of the present invention also includes the topical composition having approximately 20% hydrogen peroxide at 3-6% concentration, approximately 20% apple cider vinegar, and approximately 20% of isopropyl alcohol at 70% concentration, the composition base forming 40% of the topical composition.

In accordance with an additional feature, an embodiment of the present invention also includes the topical composition having approximately 60% hydrogen peroxide at 3-6% concentration, the composition base forming 40% of the topical composition.

In accordance with a further feature, an embodiment of the present invention also includes the topical composition having approximately 60% apple cider vinegar, the composition base forming 40% of the topical composition.

In accordance with an additional feature, an embodiment of the present invention also includes the topical composition having approximately 30% hydrogen peroxide at 3-6% concentration and approximately 30% apple cider vinegar, the composition base forming 40% of the topical composition.

In accordance with an additional feature, an embodiment of the present invention also includes the topical composition having approximately 30% isopropyl alcohol at 70% concentration, approximately 30% apple cider vinegar, and approximately 40% tea tree oil at 100% concentration, the composition base forming 10% of the topical composition.

In accordance with yet another feature, an embodiment of the present invention also includes approximately 16.66% bleach having a fragrance agent, approximately 16.66% of isopropyl alcohol at 70% concentration, and approximately 16.66% of hydrogen peroxide at 3-6% concentration.

In accordance with a further feature, an embodiment of the present invention also includes approximately 25% bleach having a fragrance agent, approximately 25% apple cider vinegar, and approximately 25% of isopropyl alcohol at 70% concentration. In additional embodiments, tea tree oil at 100% concentration is included in the topical composition and having a measurement of 3-5 cc

In accordance with yet another feature, an embodiment of the present invention also includes 0.5 oz of Butyspermum Parkii shea butter (which are derived from shea nuts).

In accordance with yet another feature, an embodiment of the present invention also includes approximately 50% hydrogen peroxide at 3-6% concentration.

In accordance with an embodiment of the present invention a topical composition for treating fungi forming on a user's body is disclosed that includes composition base formed with approximately 25% bleach having a fragrance agent, approximately 25% apple cider vinegar, approximately 25% of isopropyl alcohol at 70% concentration, and approximately 25% tea tree oil at 100% concentration and having a measurement of 3-5 cc.

In accordance with an embodiment of the present invention a topical composition for treating fungi forming on a user's body is disclosed that includes a composition base formed with approximately 25% bleach having a fragrance agent, approximately 25% hydrogen peroxide at 3-6% concentration, approximately 25% of isopropyl alcohol at 70% concentration, and approximately 25% tea tree oil at 100% concentration and having a measurement of 3-5 cc.

Although the invention is illustrated and described herein as embodied in a composition and method of treating fungi forming on a user's foot and/or nails, it is, nevertheless, not intended to be limited to the details shown because various modifications and structural changes may be made therein without departing from the spirit of the invention. Additionally, well-known elements of exemplary embodiments of the invention will not be described in detail or will be omitted so as not to obscure the relevant details of the invention.

It is to be understood that the disclosed embodiments herein are merely exemplary of the invention, which can be embodied in various forms. Therefore, specific structural and functional details disclosed herein are not to be interpreted as limiting, but merely as a basis for future claims and as a representative basis for teaching one of ordinary skill in the art to variously employ the present invention in virtually any appropriately detailed structure. Further, the terms and phrases used herein are not intended to be limiting; but rather, to provide an understandable description of the invention.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of any container retaining or housing said composition.

If any figures are incorporated in and form part of the specification, they would serve to further illustrate various embodiments and explain various principles and advantages all in accordance with the present invention.

While the preferred and other embodiments of the invention are described herein, the invention can be provided in several shapes, sizes, combinations of features and components, and varying numbers and functions of the components.

### DETAILED DESCRIPTION OF INVENTION

The invention described herein provides a composition and method of treating fungi forming on a user's foot and/or nails that overcomes known disadvantages of those known devices and methods of this general type.

More specifically, a unique composition and method of treating fungi forming on a user's foot and/or nails is disclosed, but is not necessarily limited to a user's foot or nails. For example, in some embodiments the composition may be effective to treating fungi forming on other parts of a user's body.

The method of treatment may first begin by providing a fungi treating composition having a 2-Octyl cyanoacrylate (which is a cyanoacrylate ester typically used as a wound closure adhesive, or may also be referred to as a tissue adhesive), benzethonium chloride (also known as hyamine, which is a synthetic quaternary ammonium known for its surfactant, antiseptic, and anti-infective properties), 70% isopropyl alcohol and hydrogen peroxide at 3% concentration, or a combination of the above. Preferably, however, the composition will have only one of the above base components in a small volumetric or mass fraction, e.g., approximately 5-55%, but preferably 20-50% forming the composition base of the topical composition. In one embodiment, approximately 50% efinaconazole at 5-10% concentration is also utilized in the topical composition, which is potent antifungal agent against dermatophytes, as well as activity against some molds.

Although various compositional percentages are discussed herein, any remaining components or percentages of the mixture may include one or more of the following (1) an unfiltered unpasteurized non-GMO organic apple cider vinegar, (2) an unfiltered unpasteurized non-GMO organic apple cider vinegar 100% essential tea tree oil, (3) a lavender bleach, and/or other components and ingredients set forth herein and/or non-active fillers (e.g., water), binders, or agents known to those of skill in the art (e.g., emulsifiers such as butylene glycol (also a humectant), polysorbates glyceryl stearate (also an emollient and adds pearlescence), cetearyl, cetyl, and stearyl alcohol, stearic acid, PEG (polyethylene glycol for example, PEG-100 stearate), triethanolamine, palmitates, and ceteareth 20.

In some embodiments, one fungi treating composition includes efinaconazole 5% solution or 10% concentration within the mixture of approximately 50%, wherein the composition is applied topically on the user and treats fungi or onychomycosis. The fungi treating composition may also include benzethonium chloride of 0.2% concentration within the mixture of approximately 50%. Further, in some embodiments, the percentage(s) discussed herein may be limited to the only constituent ingredients within the composition mixture, wherein the term “consisting” may be utilized to reflect the same.

In another embodiment, one fungi treating composition includes efinaconazole 5% solution or 10% concentration within the mixture of approximately 50%, wherein the composition is applied topically on the user. The fungi treating composition may also include 2-OCTYL-cyanoacrylate within the mixture of approximately 50%.

In another embodiment, one fungi treating composition includes a scented or fragranced bleach, e.g., lavender bleach sold under the brand name Clorox®, within the mixture of approximately 16.66% (i.e., +/−5%). The ingredients may consist of water ,sodium hypochlorite, sodium hydroxide, sodium carbonate poly(dialyldimethylammonium chloride), fragrance, sodium xylene sulfonate and sodium polyacrylate. The fungi treating composition may also include hydrogen peroxide 3-6% (inactive ingredient water) within the mixture of approximately 16.66%. The fungi treating composition may also include isopropyl alcohol 70% (inactive ingredient water) within the mixture of approximately 16.66%. The fungi treating composition may also include Isopropyl alcohol 70 (inactive ingredient water) within the mixture of approximately 16.66%. The fungi treating composition may also include benzethonium chloride 0.2% (inactive ingredient water) within the mixture of approximately 50%.

In another embodiment, one fungi treating composition includes a scented or fragranced bleach, e.g., lavender bleach sold under the brand name Clorox®, within the mixture of approximately 16.66%. The ingredients may consist of water ,sodium hypochlorite, sodium hydroxide, sodium carbonate poly(dialyldimethylammonium chloride), fragrance, sodium xylene sulfonate and sodium polyacrylate. The fungi treating composition may also include hydrogen peroxide 3% (inactive ingredient water) within the mixture of approximately 16.66%. The fungi treating composition may also include isopropyl alcohol 70% (inactive ingredient water) within the mixture of approximately 16.66%. The fungi treating composition may also include 2-OCTYL-cyanoacrylate within the mixture of approximately 50%.

In another embodiment, one fungi treating composition includes apple cider vinegar (inactive ingredient water) within the mixture of approximately 25%. The apple cider vinegar is preferably, but not necessarily, organic. A scented or fragranced bleach, e.g., lavender bleach sold under the brand name Clorox®, may also be utilized within the mixture of approximately 25%. A scented or fragranced bleach, e.g., lavender bleach sold under the brand name Clorox®, may also be utilized within the mixture of approximately 25%. The fungi treating composition may also include isopropyl alcohol 70% (inactive ingredient water) within the mixture of approximately 25%. The fungi treating composition may also tea tree oil 100% (preferably, but not necessarily, organic melaleuca ternifolia) within the mixture of approximately 25%.

In another embodiment, one fungi treating composition includes hydrogen peroxide 3% within the mixture of approximately 25%. A scented or fragranced bleach, e.g., lavender bleach sold under the brand name Clorox®, may also be utilized within the mixture of approximately 25%. A scented or fragranced bleach, e.g., lavender bleach sold under the brand name Clorox®, may also be utilized within the mixture of approximately 25%. The fungi treating composition may also include isopropyl alcohol 70% (inactive ingredient water) within the mixture of approximately 25%. The fungi treating composition may also tea tree oil 100% (preferably, but not necessarily, organic melaleuca ternifolia) within the mixture of approximately 25%.

In another embodiment, one fungi treating composition includes apple cider vinegar (inactive ingredient water) within the mixture of approximately 25%. The apple cider vinegar is preferably, but not necessarily, organic. A scented or fragranced bleach, e.g., lavender bleach sold under the brand name Clorox®, may also be utilized within the mixture of approximately 25%. A scented or fragranced bleach, e.g., lavender bleach sold under the brand name Clorox®, may also be utilized within the mixture of approximately 25%. The fungi treating composition may also tea tree oil 100% (preferably, but not necessarily, organic melaleuca ternifolia) within the mixture of approximately 12.5%. The fungi treating composition may also include hydrogen peroxide 3% within the mixture of approximately 12.5%. The fungi treating composition may also include 2-OCTYL-cyanoacrylate or benzethonium chloride 0.2% (inactive ingredient water) within the mixture of approximately 25%.

In another embodiment, one fungi treating composition includes isopropyl alcohol 70% (inactive ingredient water) within the mixture of approximately 25%. The apple cider vinegar is preferably, but not necessarily, organic. A scented or fragranced bleach, e.g., lavender bleach sold under the brand name Clorox®, may also be utilized within the mixture of approximately 25%. A scented or fragranced bleach, e.g., lavender bleach sold under the brand name Clorox®, may also be utilized within the mixture of approximately 25%. The fungi treating composition may also tea tree oil 100% (preferably, but not necessarily, organic melaleuca ternifolia) within the mixture of approximately 12.5%. The fungi treating composition may also include hydrogen peroxide 3% within the mixture of approximately 12.5%. The fungi treating composition may also include 2-OCTYL-cyanoacrylate or benzethonium chloride 0.2% (inactive ingredient water) within the mixture of approximately 25%.

In another embodiment to be utilized as heal balsam for cracked heels and fungal heel skin, the treating composition includes a composition base formed with approximately 50% of Butyspermum Parkii shea butter and approximately 50% tea tree oil at 100% concentration.

The above-described compositions may be topically applied to user's skin or targeted area with fungi in single or period treatments (e.g., once, twice, or three times every day) until the fungi subsides. In one embodiment, the application of the composition on fungi causes curing of the same and/or fading of the same in approximately 3 months or earlier.

Although a specific order of executing the process steps, the order of executing the steps may be changed relative to the order shown in certain embodiments. Also, two or more steps shown in succession may be executed concurrently or with partial concurrence in some embodiments. Certain steps may also be omitted for the sake of brevity. In some embodiments, some or all of the process steps can be combined into a single process.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the above described features.

What is claimed is:

1. A topical composition for treating fungi forming on a user's body comprising:
a composition base forming 10-50% of the topical composition and selected from one of the following: benzethonium chloride at 0.2% concentration and 2-Octyl cyanoacrylate.

2. The topical composition according to claim 1, further comprising:
60% efinaconazole at 5-10% concentration and with the composition base forming 40% of the topical composition.

3. The topical composition according to claim 1, further comprising:
approximately 20% hydrogen peroxide at 3-6% concentration, approximately 20% apple cider vinegar, and approximately 20% of isopropyl alcohol at 70% concentration, the composition base forming 40% of the topical composition.

4. The topical composition according to claim 3, further comprising:
approximately 60% hydrogen peroxide at 3-6% concentration, the composition base forming 40% of the topical composition.

5. The topical composition according to claim 4, further comprising:
approximately 60% apple cider vinegar, the composition base forming 40% of the topical composition.

6. The topical composition according to claim 1, further comprising:
approximately 30% hydrogen peroxide at 3-6% concentration and approximately 30% apple cider vinegar, the composition base forming 40% of the topical composition.

7. The topical composition according to claim 1, further comprising:
approximately 30% isopropyl alcohol at 70% concentration, approximately 30% apple cider vinegar, and approximately 40% tea tree oil at 100% concentration, the composition base forming 10% of the topical composition.

8. The topical composition according to claim 6, further comprising:
tea tree oil at 100% concentration and having a measurement of 3-5 cc.

9. The topical composition according to claim 1, further comprising:
approximately 25% bleach having a fragrance agent, approximately 25% hydrogen peroxide at 3-6% concentration, and approximately 25% of isopropyl alcohol at 70% concentration.

10. The topical composition according to claim 1, further comprising:
approximately 25% bleach having a fragrance agent, approximately 25% apple cider vinegar, approximately 12.5% of hydrogen peroxide at 3-6% concentration, and approximately 12.5% tea tree oil at 100% concentration and having a measurement of 3-5 cc .

11. The topical composition according to claim 1, further comprising:
approximately 25% bleach having a fragrance agent, approximately 25% isopropyl alcohol at 70% concentration, approximately 12.5% of hydrogen peroxide at 3-6% concentration, and approximately 12.5% tea tree oil at 100% concentration and having a measurement of 3-5 cc .

12. The topical composition according to claim 1, further comprising:
0. 5 oz of Butyspermum Parkii shea butter.

13. The topical composition according to claim 1, further comprising:
approximately 50% hydrogen peroxide at 3-6% concentration.

14. A topical composition for treating fungi forming on a user's body comprising:
a composition base formed with approximately 50% of Butyspermum Parkii shea butter and approximately 50% tea tree oil at 100% concentration.

15. The topical composition according to claim 14, wherein the tea tree oil has a measurement of 3-5 cc.
